(19) United States
(12) Patent Application Publication
(10) Pub. No.: US 2014/0315186 A1
(43) Pub. Date: Oct. 23, 2014

(54) IN-VITRO DIAGNOSTICS FOR CAPRINE ARTHRITIS-ENCEPHALITIS VIRUS ANTISERA AND OTHER VIRAL INTERFERENCE RELATED AGENTS

(71) Applicant: SAM CHACHOUA, Melbourne (AU)

(72) Inventor: SAM CHACHOUA, Melbourne (AU)

(21) Appl. No.: 13/865,771

(22) Filed: Apr. 18, 2013

(51) Int. Cl. G01N 33/68; C12Q 1/70

(52) U.S. Cl. CPC G01N 33/6854; C12Q 1/70
USPC 435/5; 435/235.1

(57) ABSTRACT

Various postulates and methodologies in using Caprine Arthritis Encephalitis Virus and other related agents to diagnose and treat HIV/AIDS as well as other related and/or non-related diseases are declared.

### BACKGROUND OF THE INVENTION

1. Field of the Invention

Embodiments of the present invention relate, in general, to viral interference and more particularly to in-vitro diagnostic methodology and apparatus operable to identify Caprine Arthritis-Encephalitis Virus antisera.

2. Relevant Background

Human immunodeficiency virus (HIV), a lentivirus and member of the retrovirus family, is the etiological agent for acquired immunodeficiency syndrome (AIDS). HIV remains one of the most important global public health problems. A retrovirus is an RNA virus that is duplicated in a host cell using the reverse transcriptase enzyme to produce DNA from its RNA genome. The DNA is then incorporated into the host's genome by an integrase enzyme. The virus thereafter replicates as part of the host cell's DNA. Retroviruses are enveloped viruses that belong to the viral family Retroviridae.

An HIV infection causes a gradual depletion and weakening of the immune system. This results in an increased susceptibility of the body to infections, such as pneumonia and tuberculosis and can lead to the development of AIDS. It is estimated that in excess of 33 million people worldwide are living with HIV. HIV type 1 (HIV-1) is the predominant virus worldwide, while HIV-2 differs from HIV-1 in its lower pathogenicity and higher lever of intra-subtype strain diversity. There is currently no effective vaccine or treatment for HIV.

A retrovirus stores its nucleic acid in the form of a +mRNA (including the 5′cap and 3′PolyA inside the virion) genome and serves as a means of delivery of that genome into cells it targets as an obligate parasite, and constitutes the infection. Once in the host's cell, the RNA strands undergo reverse transcription in the cytoplasm and are integrated into the host's genome, at which point the retroviral DNA is referred to as a provirus. It is difficult to detect the virus until it has infected the host.

In most viruses, DNA is transcribed into RNA, and then RNA is translated into protein. However, retroviruses function differently—their RNA is reverse-transcribed into DNA, which is integrated into the host cell's genome (when it becomes a provirus), and then undergoes the usual transcription and translational processes to express the genes carried by the virus. So, the information contained in a retroviral gene is used to generate the corresponding protein via the sequence: RNA→DNA→RNA→protein. This extends the fundamental process in which the sequence is: DNA→RNA→protein.

Virus Interference refers to the inhibition of the replication of a virus by a previous infection with another virus. The two viruses may be unrelated, related, or identical. In some cases, virus interference may take place even if the first virus was inactivated.

Several mechanisms of interference can be distinguished. These mechanisms of interference include: (1) inactivation of cell receptors by one virus may prevent subsequent adsorption and penetration by another virus; (2) the first virus may inhibit or modify cellular enzymes or proteins required for replication of the superinfecting virus; and (3) the first virus may generate destructive enzymes or induce the cell to synthesize protective substances which prevent superinfection; (4) the first virus may generate defective interfering particles or mutants which may inhibit the replication of the infecting virus by competing with it for a protein (or enzyme) available in limited quantities; this type of viral interference has been called autointerference, and depends on a greater replicative efficiency of the defective interfering particles or mutants, compared to the infecting virus.

In essence, viruses are parasites; they have to get inside of a cell and use the building blocks of the cell itself to reproduce. If two different viruses try to replicate in the same cell at the same time, they may wind up competing with each other for the building blocks, which can slow or stop their replication.

Caprine Arthritis-Encephalitis Virus (CAEV) interferes with HIV. CAEV is a single-stranded, icosahedral, RNA virus (retrovirus) of the family Retroviridae and, like HIV, the sub-family Lentivirinae. This CAEV virus is magnesium-dependent and has a RNA-dependent DNA polymerase (reverse transcriptase. Surface glycoproteins of HIV-1 and CAEV share structural regions essential for viral adsorption and for induction of neutralizing antibodies. Humans are not susceptible to CAEV infection. But as described above one lentivirus to which humans are clearly susceptible is HIV, or the AIDS virus.

Blood cells possess a higher affinity for CAEV than HIV. In essence in a competition between HIV and CAEV for the same cell CAEV will normally win. But as mentioned, CAEV is nonpathogenic in humans. Accordingly CAEV will infect cells that have not already been infected by HIV and as a result all arrest the progress of HIV. In such a sense the infection of CAE the acts as a vaccine to HIV with respect to unaffected cells. And it can be shown that CAEV creates an auto immune response that can also penetrate and destroy HIV infected cells.

As is well known, the body in the presence of a virus produces antibodies. An antibody, also known as an immunoglobulin, is a large Y-shaped protein produced by B-cells that is used by the immune system to identify and neutralize foreign objects such as bacteria and viruses. The antibody recognizes a unique part of the foreign target, called an antigen. Each tip of the “Y” of an antibody contains a paratope (a structure analogous to a lock) that is specific for one particular epitope (similarly analogous to a key) on an antigen, allowing these two structures to bind together with precision. Using this binding mechanism, an antibody can tag a microbe or an infected cell for attack by other parts of the immune system, or can neutralize its target directly (for example, by blocking a part of a microbe that is essential for its invasion and survival). The production of antibodies is the main function of the humoral immune system.

Assay systems capable of detecting the presence or absence of antibodies generated in response to the presence of viral antigens are well known. Such assay systems have proved useful in, inter alia, the diagnosis of various disease and infectious states, for example, acquired immune deficiency syndrome (AIDS), AIDS related complexes (ARC or pre-AIDS), T-lymphocytic 20 leukemia, and T-lymphocytic lymphoma.

Known assay systems, which in one example employ antibodyantigen binding, ordinarily are designed to detect solely the presence or absence of IgG (immunoglobulin G). The appearance of detectable IgG directed to antigens 25 in an infected/immunized individual, in many instances, does not occur until 30-40 days after initial infection. Typically, the IgG class antibodies are often present for months or years after infection or immunization with a foreign agent, such as a virus.

As previously discussed HIV and CAEV share structural characteristics. It has been shown that the antibodies related to HIV are likely to cross react with other retroviruses such as the MP-virus, the FPL virus and the CAE virus. The same is true in reverse in which CAEV and other non-pathogenic retrovirus antisera (antibodies) cross-react with the blood from patients with HIV. Given this similarity in activity any response, these non-pathogenic retrovirus antisera can be used to interfere with and or compete with the HIV process.

What is lacking is a definitive test for the presence of the CAEV antibody in humans. Once identified blood containing CAEV antibodies can be used in transfusions with individuals having HIV arresting the HIV progress and in some instances treating the HIV infected cells. Moreover the CAEV antibody can be isolated in the form of gamma globulin and used as an HIV treatment. What is needed, among other things, is an in-vitro diagnostic device that can definitively identify CATV antibodies in human beings. These and other deficiencies of the prior art are addressed by one or more bonds of the present invention.

### DESCRIPTION OF THE INVENTION

Described hereafter by way of example, or one or more embodiments relating to the viral interference of HIV and CAEV. Through the phenomena known as viral interference the replication in advance of HIV in humans can be reduced or arrested by using the viral interference of CAEV. Moreover antibodies produced by the infection of CAEV can be used to treat individuals previously infected by HIV without having to be directly exposed the CAE virus. In one embodiment of the present invention CAEV antisera is identified by using an in vitro diagnostic tool. Once the presence of CAEV antisera or CAEV itself has been identified in the donor the antisera and/or the cave of strain most effective at interfering with HIV can be extracted.

Embodiments of the present invention are hereafter described in detail with reference to the accompanying Figures. Although the invention has been described and illustrated with a certain degree of particularity, it is understood that the present disclosure has been made only by way of example and that numerous changes in the combination and arrangement of parts can be resorted to by those skilled in the art without departing from the spirit and scope of the invention.

The following description with reference to the accompanying drawings is provided to assist in a comprehensive understanding of exemplary embodiments of the present invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. Also, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Upon reading this disclosure, those of skill in the art will appreciate still additional alternative structural and functional designs for a system and a process for an interaction system for a distributed tangible user interface through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The CAE virus in humans is non-pathogenic. Accordingly human blood is not currently screened or tested for the CAE virus. The CAE virus can be identified by looking for the CAE antibody or its polymerase chain reaction, PCR. CAEV can also be identified in conjunction with an autoimmune activity. Accepting for arguments sake that the CAE virus interferes with HIV, a simple and high level test for the determination of CAEV in blood is to introduce HIV into a donor sample and observe the growth or destruction of HIV. Such an observance does not conclusively identify what component of the blood is producing an HIV resistance but it does identify that the donor sample is HIV resistant.

According to one embodiment of the present invention, HIV resistant blood is identified through a variety of screening processes. One component of that screening process, is to identify the presence of the CAEV antibody or that the blood has had prior exposure to CAEV. The process may also include identifying that the blood possesses autoimmune features to other diseases such as Lupis, or mixed connective tissue disease and other diseases that give resistance to HIV.

However most blood does not possess resistance to HIV. Accordingly one embodiment of the present invention is a test kit using a variety of parameters to identify blood that is HIV resistant. The kit will identify antibodies that are resistant to HIV including CAEV antibodies, measles antibodies, Feline Immune Deficiency Virus, etc. Many viral antibodies exhibit cross resistance to HIV. Some are retro-viruses while others are not.

It has been shown that HIV goes into remission upon the introduction of other certain viruses. For example a child with HIV will find that the HIV is in remission once infected with measles. CAEV has been found to highly interfere with HIV yet there are no current tests by which to identify CAEV antisera in humans.

According to one embodiment of the present invention a test kit that identifies CAEV in human donors includes a system identifying PCR and serum antibodies. In addition lymphocytes are removed from the human being and are specifically tested for CAEV content; live CAEV or evidence of CAEV exposure. The test identifies strains that offer transient resistance as well as those strains that leave remnants of the CAEV in the white blood cells. To do so the cells are broken apart by sonification or lysis or other method to extract DNA and RNA from the white blood cell. Once the RNA and DNA have been extracted they are tested for CAEV fragments.

Thereafter only those white blood cells in which HIV infects are examined. This is done, in one embodiment, by flow cytometry to identify the CD4 and CD8, These can be then broken apart and from them extract out the RND/DNA and determine if there is CAEV fragments. If there is CAEV fragments in the CD4 or CD8 it serves as evidence that the donor has had long term infection or chronic infection of CAEV. Blood possessing these fragments of CAEV in the RNA/DNA cannot be infected by HIV.

In this case the target cells of HIV are already infected by CAEV and are not receptive to HIV. Cells with the CAEV fragment present in a latent phase will not identified by a normal test. The test of the present invention identifies that CAEV has been incorporated into the DNA genome.

One object of the present invention is a test to identify blood that possesses latent or CAEV fragments; that is that the DNA genome of the donor blood contains CAEV.

According to another embodiment of the present invention individuals can be infected with CAEV prior to blood donation. That injection will ultimately produce blood with a CAEV fragment in the donor DNA. It is counter intuitive to adulterate blood with prior to using it for a donation. CAEV normally has no interaction in human beings and thus there is generally no reason to vaccinate a human for a disease that is only pathogenic in animals. One embodiment of the present invention is to vaccinate human donors with a CAEV vaccine or to infect them with CAEV so their own autoimmune system can generate antibodies to the CAEV. Blood donated from these individuals will be HIV resistant. One embodiment of the present invention is the creation and use of a CAEV vaccine suitable for human application.

In animals a CAEV vaccine has limited effectiveness just at a HIV vaccine based on HIV has limited effectiveness. The virus actually is benefited by the immune response. But according to one embodiment of the present invention a first, unique, disease state is used to treat a second, unique, disease state. In this case a CAEV disease state is used to treat a HIV disease state.

CAEV as a vaccine in human beings is not used to prevent CAEV but rather used to induce an infection of CAEV and as a result prevent another disease, HIV.

This is a trans-species infection to create resistance against another disease state. CAEV has no affect on humans.

In this case CAEV and HIV are different and while the immune response to CAEV may provide resistance to HIV, the presence of the CAEV infection itself inhibits HIV. It is true that the virus will create an immune response and that this immune response is therapeutic. But a virus can occupy a cell so that it cannot be further infected. An immune response will attack a virus cell while circulating but the virus, in this case CAEV will occupy the lymphocytes so that HIV can never penetrate the cell.

Certain infections of CAEV are subclinical while others are overt. Overt infections are more aggressive and more likely to displace HIV from its locus. After infection HIV becomes incorporated into the DNA. The only way to displace it is to replace it with a stronger virus. CAEV displaces HIV.

Accordingly to one embodiment of the present invention an in-vitro diagnostic device and protocol identifies overt, aggressive strains of CAEV that are effective at displacing HIV in the human genome. According to one embodiment, blood identified as having been infected with CAEV is mixed with blood that is infected with HIV. Thereafter the viral effect if observed. If HIV in the mixed blood is diminished the CAEV blood used in the test possesses an aggressive strain of CAEV. The blood is not only compatible but is therapeutic to the treatment of HIV. For example, more than just being preventative, meaning that the HIV invention does not increase, this CAEV infected blood actually treats that HIV and reduces its presence.

This identified blood has a CAEV aggressive nature. Then a further step can be accomplished to show that it has gene loci and protein overlap. Moreover you can conduct a gene map sequencing and protein map sequencing to verify the overlap. The greater the overlap the greater the likelihood that the strain will work. The test that shows that the virus is chronically active and aggressive is simply observing the effect of the mixed blood samples. The blood samples having the greatest overlap will produce the most aggressive nature but the overlap will likely vary from one donor to another. So an individual HIV DNA map can be cross sequenced with CAEV infected blood to identify what donor blood will be the most effective against that particular HIV patient.

According to another embodiment of the present invention, only diseased cells are effected by the introduction of CAEV. Through the use of viral reconstitution diseased cells can be targeted. Virus can be broken apart and reconstituted. One aspect of the present invention is to use specific viral fractions that can be isolated from lymphocytes or other issue after chronic CAEV infection in humans or blood donors can be reactivated by HIV.

Viruses have the capacity to reconstitute with the correct fragment. Alone however these fragments are harmless as they are incomplete. Once it finds a virus or RNA fragment that is infected with HIV, the CAEV virus will rebuild itself. Then that new virus will seek out and find HIV virus. It will produce absolute displacement of HIV. Thus CAEV is not active but will only reactivate once HIV is introduced.

One embodiment of the present invention is the identification of the CAEV strain that can be broken into fragment so that these fragments can be introduced into humans. These strains are identified by co-culturing HIV and CAEV, limiting food supply and supplying a mutating capability by introducing x-ray, or chemical techniques to create a competitive environment. The result is a culture that eliminates the HIV in the culture.

The soluble fragments of CAEV that you isolate from white blood cells of a person exposed to an animal that has a server CAEV infection will aggressively seek out and destroy HIV. This stain easily outperforms HIV. For example you can infect an animal with CAEV and then introduce HIV. The CAEV strain will be enhanced to attack HIV. This will optimize the CAEV strain.

This concept of viral interference can be applied to other viruses beyond CAEV. Other viruses are also resistant to HIV. These other viruses can be identified and use to develop other HIV resistant virus that can be used in isolation or combined with CAEV.

According to another embodiment of the present invention gamma globulin of the CAEV inflected blood is isolated for application to HIV patients. This gamma globulin will be resistant and therapeutic to HIV.

As will be understood by those familiar with the art, the invention may be embodied in other specific forms without departing from the spirit or essential characteristics thereof. Likewise, the particular naming and division of the modules, managers, functions, systems, engines, layers, features, attributes, methodologies, and other aspects are not mandatory or significant, and the mechanisms that implement the invention or its features may have different names, divisions, and/or formats. Furthermore, as will be apparent to one of ordinary skill in the relevant art, the modules, managers, functions, systems, engines, layers, features, attributes, methodologies, and other aspects of the invention can be implemented as software, hardware, firmware, or any combination of the three. Of course, wherever a component of the present invention is implemented as software, the component can be implemented as a script, as a standalone program, as part of a larger program, as a plurality of separate scripts and/or programs, as a statically or dynamically linked library, as a kernel loadable module, as a device driver, and/or in every and any other way known now or in the future to those of skill in the art of computer programming Additionally, the present invention is in no way limited to implementation in any specific programming language, or for any specific operating system or environment. Accordingly, the disclosure of the present invention is intended to be illustrative, but not limiting, of the scope of the invention.

I claim:

1. Caprine Arthritis Encephalitis Virus or “CAEV” (a single-stranded, icosahedral, RNA virus (retrovirus) of the family Retroviridae, a sub-family Lentivirinae, that is magnesium-dependent and has a RNA-dependent DNA polymerase (reverse transcriptase with surface glycoproteins of HIV-1 and CAEV share structural regions essential for viral adsorption and for induction of neutralizing antibodies), at which humans are not susceptible to CAEV infection,
a. can out-compete HIV in cell infection (due to blood cells having a higher affinity for CAEV than HIV) but has no pathogens to infect humans, because CAEV share similar structural characteristics and can cross-react with blood from patients with HIV;
b. can supplant HIV after it has infected the host—CAEV is a stronger virus that can displace HIV that has previously infected the person and where such HIV has been incorporated into the human DNA.
c. can create an auto immune response that can also penetrate and destroy HIV infected cells, in which one mechanism is the creation of an antibody-immunoglobulin that targets antigens using the paratope on the antibody specific to the epitope on the HIV-antigen, to bind such antigen for prevention of further infection by the antigen and for subsequent destruction by the human immune system
d. Can interfere with HIV's infection of human cells

2. In claim 1d above, several mechanisms of interference can be distinguished such as
a. Inactivation of cell receptors by one virus may prevent subsequent adsorption and penetration by another virus; and/or
b. The first virus may inhibit or modify cellular enzymes or proteins required for replication of the superinfecting virus; and
c. The first virus may generate destructive enzymes or induce the cell to synthesize protective substances which prevent super-infection; and/or
d. The first virus may generate defective interfering particles or mutants which may inhibit the replication of the infecting virus by competing with it for a protein (or enzyme) available in limited quantities; this type of viral interference has been called auto-interference, and depends on a greater replicative efficiency of the defective interfering particles or mutants, compared to the infecting virus.

3. Resistance to HIV can be detected by diagnosing for the presence of
a. CAEV antibody,
b. Measles Antibodies,
c. Feline Immune Deficiency virus; or
d. Presence of autoimmune features to other diseases such as Lupus or Mixed Connective Tissue Diseases or other such diseases

4. In claim 3a above, CAEV antibody can be isolated in the form of gamma globulin and can be used in the prevention and treatment of HIV.

5. In claim 3a above, CAEV antisera can be identified using an in vitro diagnostic tool by looking for CAE antibody or its polymerase chain reaction.

6. In claim 5 above, test kits that identify CAEV in human donors include identifying PCR and serum antibodies or human Lymphocytes can be tested for CAEV content or CAEV exposure.

7. Methods to identify strains that offer resistant to HIV can include but not limited to
a. Tests that identify strains that offer transient resistance to HIV can be accomplished by sonification or lysis of white blood cells to extract its DNA/RNA to test for fragments of CAEV by but not limited to testing by flow cytometry to identify CD4 and CD8 exposed to CAEV.
b. In claim 7a above, one method to identify and isolate aggressive strains of CAEV that can displace HIV is to place CAEV infected blood with HIV infected blood to determine the viral effect.
c. In claim 7a above, the gene loci and protein overlap can be determined and sequencing can be conducted to verify overlap to verify the level of therapeutic aggressiveness.
d. An individual's HIV DNA map can be cross sequenced with CAEV infected blood to identify which donor blood will be most effective against HIV patient
e. An aggressiveness index for such strains can be created for each patient infected with HIV.

8. Only diseased cells are affected by CAEV and using viral fractions after chronic CAEV infections in humans or donors, can be reactivated using HIV and such reactivated CAEV can seek out HIV virus and displace such HIV from its infection position.

9. In claim 8 above, the author has identified a method of determining and isolating strains of CAEV that can be broken into fragments that can be introduced into humans by co-culturing HIV and CAEV, limiting food supply and supplying a mutating capability through x-rays or chemical techniques to create a competitive environment, resulting in elimination of HIV from the culture.

10. In claim 8 above, infecting an animal with CAEV and then introducing HIV to such animal will create a strain of CAEV that will be enhanced to attack HIV, resulting in an optimizing of type of CAEV strain.
